(19) United States
(12) Patent Application Publication
(10) Pub. No.: US 2026/0234082 A1
(43) Pub. Date: Aug. 13, 2026

(54) LOW CONVERSION START-UP OF A HYDROCARBON CRACKING FURNACE

(71) Applicant: NOVA CHEMICALS (INTERNATIONAL) S.A., Fribourg (CH)

(72) Inventor: Michael KOSELEK, Red Deer (CA)

(21) Appl. No.: 19/471,150

(22) PCT Filed: Feb. 28, 2024
(86) PCT No.: PCT/IB2024/051924

§ 371 Date: Sep. 30, 2025

Related U.S. Application Data
(60) Provisional application No. 63/494,015, filed on Apr. 4, 2023.

(51) Int. Cl. C07C 5/327

(52) U.S. Cl. CPC C07C 5/327

(57) ABSTRACT

Methods are provided for increasing a run length of a hydrocarbon cracking furnace An exemplary method includes starting up a hydrocarbon cracking furnace at a first conversion level of a C₁-C₄ alkane, wherein the first conversion level is less than or equal to 60% conversion, and maintaining the first conversion level for a predetermined interval.

### RELATED APPLICATION

This application claims the benefit under 35 U.S.C. § 119(e) of U.S. provisional application No. 63/494,015, filed Apr. 4, 2023, which is incorporated by reference herein in its entirety.

### TECHNICAL FIELD

The present disclosure relates to the field of hydrocarbon cracking furnaces, and methods to extend the run length of a hydrocarbon cracking furnace.

### BACKGROUND ART

Hydrocarbon cracking furnaces are used to generate saleable products. The start-up of a hydrocarbon cracking furnace heater's coils, when converting lighter C₁-C₄ alkane feedstock to C₂-C₄ alkenes, tends to be done as quickly as possible to return to producing the maximum amount of saleable products. When a hydrocarbon cracking furnace enters the feed-in block of the start-up procedure, once the alkane feed is fully admitted, often the operator tries to achieve the maximum alkene production rate from the hydrocarbon cracking furnace. This production rate is typically achieved by raising the combined coil output temperature (COT) to the temperature at which the desired conversion, typically about 65% or greater, is obtained.

Hydrocarbon cracking furnaces must have their runs terminated regularly for decoking. Historically, the decoke time has been kept as a constant, and the return to cracking must be as close to instantaneous as possible. The practice of starting a hydrocarbon cracking furnace at maximum conversions does return the hydrocarbon cracking furnace to making the maximum amount of products in the shortest time possible. This has been the accepted best practice in the industry since the early days of cracking facilities.

### SUMMARY OF INVENTION

An embodiment described in examples herein provides a method for extending a run length of a hydrocarbon cracking furnace. The method includes starting up a hydrocarbon cracking furnace at a first conversion level of a C₁-C₄ alkane, and maintaining the first conversion level for a predetermined interval. The first conversion level is less than or equal to 60% conversion.

In an aspect, the predetermined interval is between about 1 day and about 75 days. In an aspect, the predetermined interval is at least 3 days. In an aspect, the predetermined interval is selected according to furnace design.

In an aspect, the first conversion level is in a range of about 50% to about 58%. In an aspect, the first conversion level is in a range of about 55% to about 58%.

In an aspect, the C₁-C₄ alkane comprises ethane. In an aspect, the C₁-C₄ alkane is converted to ethylene.

In an aspect, the method further includes increasing the first conversion level of the C₁-C₄ alkane to a second conversion level, wherein the second conversion level is greater than or equal to about 61%.

In an aspect, increasing the first conversion level to the second conversion level includes iterating between increasing the first conversion level by about 0.5 to about 2% over a period of time in a range of about 2 hours to about 24 hours; and holding for a hold time in a range of about 12 hours and about 36 hours.

In an aspect, the second conversion level is in a range of about 61% to about 75%. In an aspect, the second conversion level is about 65%.

In an aspect, a run length of the furnace is at least 90 days. In an aspect, a run length of the furnace is at least 150 days. In an aspect, a run length of the furnace is at least 400 days.

In an aspect, a run length of the furnace is extended by at least 25% as compared to a run length of the method performed without starting up at the first conversion level. In an aspect, a run length of the furnace is extended by at least 100% as compared to a run length of the method performed without starting up at the first conversion level. In an aspect, a run length of the furnace is extended by at least 200% as compared to a run length of the method performed without starting up at the first conversion level.

### BRIEF DESCRIPTION OF DRAWINGS

FIG. 1 is a depiction of the effect of decoking procedure on the production days for a furnace.

FIG. 2 is a flow chart of an example method 200.

FIG. 3 is a time plot of a comparative example method and an example method performed on a furnace.

FIG. 4 is a time plot of example methods performed on a furnace.

FIG. 5 is a time plot of comparative example methods and example methods performed on a furnace.

FIG. 6 is a time plot of an example method performed on a furnace.

FIG. 7 is a time plot of an example method showing the conversion levels, ethylene make, and fouling rate, as well as the fouling rate for a comparative method with a high conversion start up.

### DESCRIPTION OF EMBODIMENTS

The typical practice of starting up a hydrocarbon cracking furnace at the desired conversion level to maximize C₂-C₄ alkene production can be detrimental to the run length of the furnace. The main problem associated with high conversion start-up procedures is the high rate of hydrocarbon byproducts that build up within the furnace coil. During startup of the furnace, the inner surface of the coil has a high activity and heat transfer rate, which leads to the formation of a significant amount of high boiling point byproducts that accumulate in the coil. The dehydrogenation of these byproducts results in a build-up of undesirable coke-like matter that raises the back pressure on the coil.

In order to remove this fouling matter from the coil, a decoking procedure must be carried out regularly. The decoking procedure is performed by burning out the fouling matter at high temperatures in the presence of oxygen. Depending on the style of heater, typical run lengths for a hydrocarbon cracking furnace are in the range of about 4 days to about 60 days before the furnace must be taken offline for decoking. For example, the hydrocarbon cracking furnace may stay online for about 25-35 days, and then be subject to decoking for a period of 2-4 days.

Due to the frequent need to perform decoking procedures to remove fouling matter, drawbacks to starting up the furnace at a high conversion level include lost production during the decoking procedure and a need for extra hydrocarbon cracking furnaces to make up for the lost plant capacity. Since decoking is typically required monthly, a hydrocarbon cracking furnace may be out of production for 35 to 50 days per year. In atypical 10-furnace plant design, this would necessitate an 11th furnace to ensure the backend of the plant was operating at full capacity. The backend of the plant includes the part of the plant that uses the products from the cracking furnace, such as a polymerization process. If the run length of hydrocarbon cracking furnaces could be extended, the production could be significantly increased.

Provided herein is a method to extend the run length of a hydrocarbon cracking furnace. The method includes starting up the furnace at a lower temperature and conversion level, which is maintained for a predetermined interval. This can be followed by increasing the conversion level to a second conversional level, rather than proceeding to the final conversion temperature upon start-up. This method can allow for a significant increase in the run length of the furnace, for example, beyond the typical 4-60 days. As used herein, the term “run length” or “run time” refers to the on-stream time of a hydrocarbon cracking furnace, which includes the feed in through to the first conversion level and second (maximum) conversion level until the feed is removed and the decoking process begins. In other words, this refers to the total amount of time the furnace is online without needing to be taken offline for decoking. For example, a furnace having a tube metal temperature (TMT) greater than or equal to 1080° C. (1976° F.) as measured by an optical pyrometer should be taken offline to enter the decoking process. The method can allow for stabilization of the inner surface of a hydrocracking furnace coil to extend the length of time that the furnace is online.

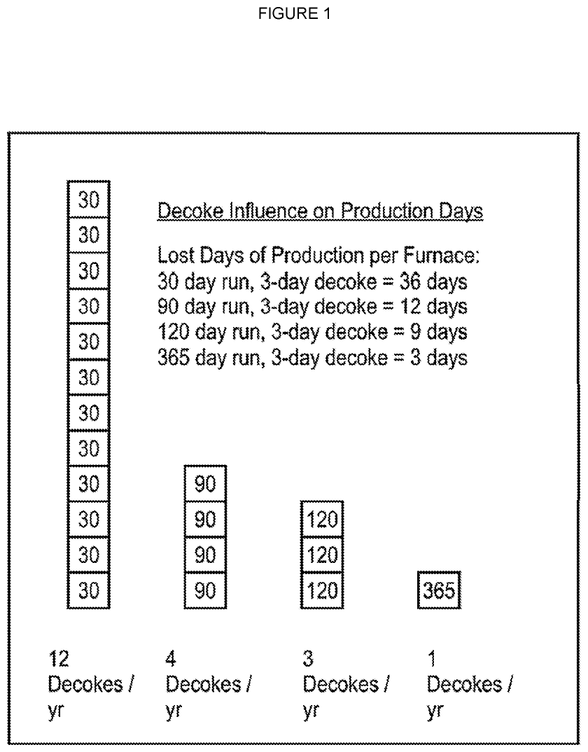

By extending the days that the furnace is online, the method disclosed herein can increase the profitability of a hydrocarbon cracking furnace. For example, FIG. 1 shows that by having a furnace last for a run length of 365 days as compared to a typical run length of 30 days, a plant could realize 33 days of extra production per furnace per year. This can bring atypical 10-furnace facility down from 360 days off-line to 30 days off-line. Depending on the amount of extra days online compared to the days spent at lower conversion rates during the implementation of the method, it can be more profitable to start up the hydrocarbon cracking furnace at a low conversion according to the method described herein.

Further, the method may reduce formation of undesirable byproducts, such as high boiling point materials, radicals, coke matter, and polycyclic aromatic hydrocarbons (PAH). Without wishing to be bound by theory, it is believed that at lower conversion levels, formation of a small amount of these byproducts on the inner surface of the hydrocarbon cracking furnace tube can provide a partially inert barrier between the tube's surface metallurgy and the feed gas, lowering the rate of byproduct formation. As used herein, the furnace “coil” refers to the total length of the metal tubes connected together from the inlet to the outlet, and the “tube” refers to the distance between two consecutive u-bends within the radiant box of the furnace.

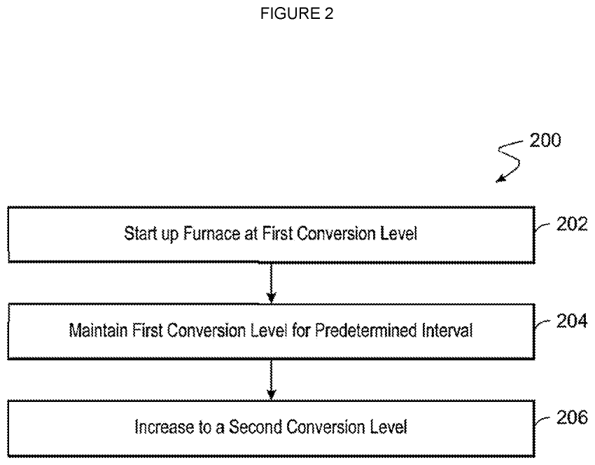

FIG. 2 is a flow chart of an example method 200. The method 200 begins at block 202, with starting up a hydrocarbon cracking furnace at a first conversion level of a C₁-C₄ alkane, wherein the first conversion level is less than or equal to 60% conversion. At block 204, the first conversion level is maintained for a predetermined interval. At block 206, the first conversion level is increased to a second conversion level, wherein the second conversion level is greater than or equal to 61% conversion. As used herein, “conversion level” refers to the percent of C₁-C₄ alkane molecules in the C₁-C₄ alkane feed that are converted to a reaction product, and can be set by adjusting a temperature of the furnace, a pressure of the furnace, or both, according to methods known in the art. Variation in feedstocks, the quality of the feedstock, and the presence of contaminants such as sulfur can also affect the conversion level, as is understood by one skilled in the art.

Startup of furnace at first conversion level.

The first conversion level of block 202 can be any suitable conversion level that is less than or equal to 60% conversion. In some embodiments, the first conversion level is in a range of about 50% to about 58%. In some embodiments, the first conversion level is in a range of about 55% to about 58%, about 50% to about 55%, or about 50% to about 57%. In some embodiments, the first conversional level is about 58%. In some embodiments, the first conversion level is about 57%. In some embodiments, the first conversion level is about 55%.

The first conversion level can be selected according to economic factors, furnace design, or both. In some embodiments, a first conversion level lower than 50% can result in the formation of more recycled and lower grade coproducts, such as lower value alkane products. Factors that may be considered regarding the furnace design include the residence time of the furnace. The length of the furnace coil determines how long a feedstock molecule will reside in the radiant zone of the furnace. In some embodiments, the furnace has a short residence time, such as less than 0.3 seconds, and the first conversion level is about 55%. In some embodiments, the furnace has a medium residence time, such as 0.3 to 0.7 seconds, and the first conversion level is about 57%. In some embodiments, the furnace has a long residence time, such as greater than 0.7 seconds to about 1.5 seconds, and the first conversion level is about 58%.

In some embodiments, the C₁-C₄ alkane of block 202 is ethane. In some embodiments, a C₁-C₄ alkane has a steam diluent. In some embodiments, the steam:oil ratio of the C₁-C₄ alkane is about 25:75 steam:C₁-C₄ alkane. In some embodiments, the C₁-C₄ alkane is converted to a C₂-C₄ alkene, such as ethylene.

Maintain conversion level for predetermined interval.

At block 204, the first conversion level is maintained for a predetermined interval. For example, the predetermined interval is greater than about twelve hours or greater than about one day (i.e., greater than about 24 hours). In some embodiments, the predetermined interval is between 5 days and 75 days, between 15 days and 30 days, between 3 days and 30 days, between 4 days and 20 days, or between 3.5 days and 5 days. In some embodiments, the predetermined interval is at least 3 days, at least 4 days, at least 4.5 days, at least 5 days, at least 7 days, at least 8 days, at least 9 days, at least 12 days, at least 15 days, at least 17 days, at least 18 days, at least 20 days, at least 22 days, at least 25 days, or at least 30 days.

The predetermined interval may be selected according to several factors, including desired productivity of the furnace, furnace design, fouling rate, and combinations thereof. For example, productivity of the furnace depends on factors including the run length of the furnace and the level of alkene production. An optimal predetermined interval may increase the run length of the furnace level while achieving a high level of alkene production. Increasing the predetermined interval can increase the run length of the furnace. In some embodiments, a predetermined interval of at least one day results in an increased run length of about 50%, as compared to a run length of the method performed without starting up at the first conversion level. In some embodiments, a predetermined interval of at least two days results in an increased run length of about 125%. In some embodiments, a predetermined interval of at least three days results in an increased run length of about 200%, such as a run length of 150 to 200 days. In some embodiments, a predetermined interval of at least four days results in a run time of over 300 days. On the other hand, increasing the predetermined interval results in a lower level of alkene production during that time, due to the low conversion level.

The predetermined interval may also be selected according to furnace design in order to achieve longer run lengths and maximum alkene yield. For example, furnace design factors include number of passes, furnace style, coil diameter, and residence time. Within a furnace, the pipes can travel either horizontally or vertically. As used herein, the “number of passes” refers to the number of u-bends in the furnace, where a pass is measured from an outlet of a u-bend to an inlet of the next consecutive u-bend. In some embodiments, the furnace has a 6″ diameter, 11-pass coil and the predetermined interval is about 17 days. In some embodiments, the furnace has a 3″ diameter, 4-pass coil and the predetermined interval is about 5 days. In some embodiments, the furnace has a 6″ diameter, 5-pass coil and the predetermined interval is about 8 days. In some embodiments, the furnace has a 3.5″ diameter, 7-pass coil and the predetermined interval is about 5 days.

The predetermined interval may be selected according to the residence time of the furnace. In some embodiments, the residence time is greater than 0.9 seconds and the predetermined interval is up to about 15 days. In some embodiments, the residence time is in a range from 0.5 second to 0.9 seconds and the predetermined interval is about 5 days. In some embodiments, the residence time is in a range from 0.2 second to 0.5 seconds and the predetermined interval is up to about 4.5 days. In some embodiments, the residence time is less than 0.2 seconds and the predetermined interval is up to about 3.5 days.

The predetermined interval can further be selected according to the fouling rate to ensure that the coil flow coefficient has reached a cresting point, as determined using the Darcy-Weisbach equation. The Darcy-Weisbach equation is

$$\frac{Δ p}{L} = {f}_{D} \cdot \frac{ρ}{2} \cdot \frac{{〈 v 〉}^{2}}{{D}_{H}} ;$$

wherein the pressure loss per length of the coil

$$\frac{Δ p}{L}$$

is a function of the density of the fluid (ρ), the hydraulic diameter of the pipe (D_{H}), the mean flow velocity ( [symbol] v [symbol] ), and the Darcy friction factor (f_{D}). As cross-sectional area is lost, the pressure must increase to get the same flow through the reduced area. The coil flow coefficient

$$( \frac{Δ p}{L} )$$

can be plotted as a function of time to determine at which point the coefficient reaches a point of curvature where it begins to level off and no longer significantly increase, referred to herein as a “cresting point”. In some embodiments, the predetermined interval is at least as long as the time at which the coil flow coefficient reaches the cresting point.

Operation of furnace at second conversion level.

At block 206, the first conversion level of the C₁-C₄ alkane in increased to a second conversion level. The second conversion level of block 206 is greater than about 61%. In some embodiments, the second conversion level is in a range between 61% and 75%. For example, the second conversion level is about 65%.

In some embodiments, the increase from the first conversion level to second conversion level is in a gradually ramped pattern. For example, block 206 can include iterating between increasing the first conversion level by about 0.5 to about 2% over a period of time in a range of about 2 hours to about 24 hours, and holding for a hold time in a range of about 12 hours and about 36 hours, until the second conversion level is reached. For example, block 206 includes iterating between increasing the first conversion level by about 1% over a period of time of about 12 hours, and holding for a hold time of about 24 hours, until the second conversion level is reached. In some embodiments, increasing to the second conversion level is performed over about 10.5 days until a second conversion level of about 65% is reached.

The method 200 can provide for an extended run length of the furnace as compared to a run length of the furnace without starting up at the first conversion level, such as compared to a run length of a method wherein the furnace is started up at an initial conversion level of 61% or greater. In some embodiments, a run length of the furnace is at least 90 days, at least 150 days, at least 300 days, at least 400 days, or at least 450 days. In some embodiments, a run length of the furnace is in a range between about 450 days and about 775 days. In some embodiments, a run length of the furnace is extended by at least 25%, at least 50%, at least 100%, at least 125%, or at least 200% as compared to a run length of the method performed without starting up at the first conversion level.

Other than in the operating examples or where otherwise indicated, all numbers or expressions referring to quantities of ingredients, reaction conditions, etc. used in the specification and claims are to be understood as modified in all instances by the term “about”. Accordingly, unless indicated to the contrary, the numerical parameters set forth in the following specification and attached claims are approximations that can vary depending upon the desired properties, which the present disclosure desires to obtain. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical values, however, inherently contain certain errors necessarily resulting from the standard deviation found in their respective testing measurements.

In addition, it should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

### Examples

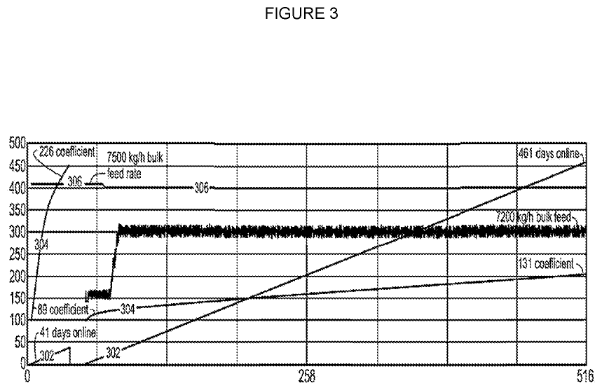

FIG. 3 shows a Time Plot of two experiments performed on a cracking furnace A, with Total Days Online 302, Coil Flow Coefficient 304, and Furnace Bulk Feed Rate 306. The Coil Flow Efficient 304 is calculated by the Darcy-Weisbach equation, and refers to the fouling rate of coke and other byproducts as it builds up in the tube inside the furnace. As time progresses, the tubes and coil build-up a level of fouling material which reduces the cross-sectional area of the tube and results in the back pressure increasing on the coil until it needs to be cleaned by the decoking process. Bulk Feed Rate 306 refers to the sum of the feedstock and dilution steam flow that proceeds through the coil in the furnace. Cracking furnace A was a Foster Wheeler natural draft furnace having a 4-coil, 11-pass, 6″ tube and a residence time of about 1.1 seconds.

The first experiment performed was a comparative example where the furnace was started up at a 65% conversion level. The Total Days Online 302 for the comparative example was 41 days before the furnace had to be taken offline for decoking due to the high level of fouling reached, as shown by Coil Flow Coefficient 304. A second experiment was performed according to the method disclosed herein, where the furnace was started up at a low conversion level, held for a predetermined interval, and then gradually increased to a higher conversion level. This example achieved 461 days online, as shown by Total Days Online 302, due to a significantly lower fouling rate, as shown by Coil Flow Coefficient 304.

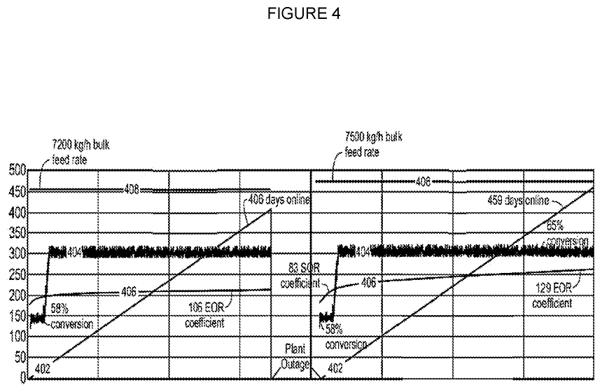

FIG. 4 shows a Time Plot of two experiments performed on cracking furnace B, with Total Days Online 402, Conversion 404, Coil Flow Coefficient 406, and Furnace Bulk Feed Rate 408. Cracking furnace B was a Foster Wheeler natural draft furnace having a 4-coil, 11-pass, 6″ tube and a residence time of about 1.1 seconds. Cracking furnace B also had two extra heat absorbing passes in the convection bank of the bulk flow system. Each experiment included starting up the furnace at a conversion level of about 58%, holding for a predetermined interval, and gradually ramping the conversion level to about 65%, as shown by Conversion 404. The fouling rate (as shown by Coil Flow Coefficient 406) was relatively low for both experiments, allowing long run lengths to be achieved. The coil flow coefficient 406 at the start of the run (SOR) and end of the run (EOR) are noted in FIG. 4. The first experiment achieved a run length of 406 days, and the second experiment achieved a run length of 459 days, as shown by Total Days Online 402. Notably the two runs shown in FIG. 3 and FIG. 4 occurred simultaneously, on opposite sides of the west to east highline pipe rack. The plant outage between the two experiments as shown in FIG. 4 refers to a plant turnaround time period wherein the plant was shut down and the furnace was evacuated of all hydrocarbon, fouled areas were serviced, and the furnace was then restarted for the second experiment. The process took about 40-60 days to complete.

Multiple experimental operating instructions (EOI) were performed on two heater technologies having an 11-pass coil design and a 4-pass coil design, across nine different heaters. When the furnace was started up after a decoking procedure at a conversion level of less than 60% and held for at least five days, run lengths of greater than 150 days were realized on a long residence time heater having a residence time of about 1.1 seconds. On a shorter residence time heater having a residence time of about 0.4 seconds, run lengths of greater than 80 days online were realized.

When the furnace was started up at a conversion level of 58% and held for 15 days on a long residence time heater having a residence time of about 1.1 seconds, run lengths were increased to about 400 days on four heaters and back-to-back greater than 450 day run lengths on a fifth heater.

On shorter residence time heaters having a residence time of about 0.4 seconds, when the furnace was started up at a conversion level of 58% and held for 20 days, run lengths increased from typical 15-30 days to greater than 150 days.

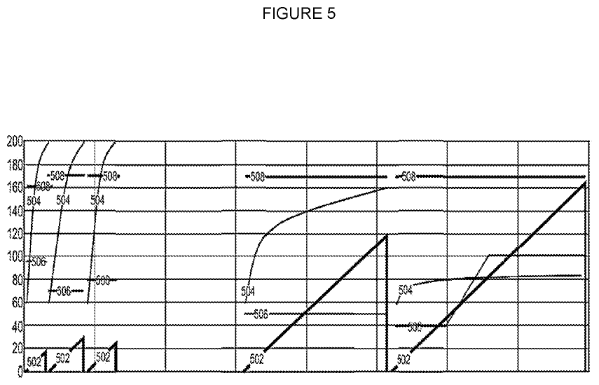

FIG. 5 shows a Time Plot of five experiments performed on cracking furnace C, with Total Days Online 502, Coil Flow Coefficient 504, Conversion 506, and Furnace Bulk Feed Rate 508. Cracking furnace C was a Stone & Webster induced draft furnace having four coils with four passes per coil, 3.5″ tubes, and a residence time of about 0.4 seconds. The first four runs were started up at a set conversion level and held there, and the fifth experiment started at a low conversion level and was ramped to a high conversion level. These experiments show that if the furnace is started at a high conversion level, such as 65%, this results in a significantly shorter run length. If the furnace is started at 60% conversion level, a longer run length and extra ethylene yield was achieved over the length of the run. If the furnace is stated at a 58% conversion level and then slowly raised, a much longer run length was achieved with an even higher ethylene yield.

The lower conversion start-up process worked on two furnaces and on six successive runs. The extended run lengths also had higher ethylene yield over the entire run versus the industry standard start-up.

A number of experiments were conducted on an 11-pass 460′ coil Foster Wheeler hydrocarbon cracking furnace. There were two main considerations for these experiments: initial ethane conversion level and duration of low conversion hold period. Typical start-ups for this style of furnace are 64-65% ethane conversion. For the EOIs, 58% conversion was chosen as the starting point.

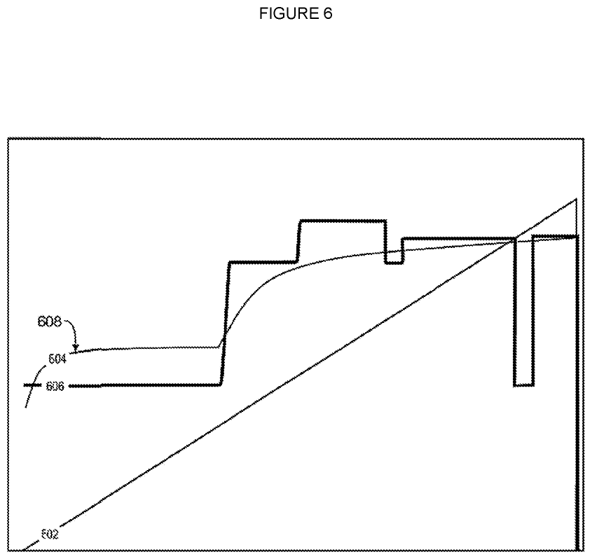

FIG. 6 shows a Time Plot of an experiment performed on the 11-pass hydrocarbon cracking furnace, with days online 602, Coil Flow Coefficient 604, and Conversion 606. The initial hold at 58% conversion duration was 59 days, vs. the expected run of 35 days. Since the coefficients had not changed, on day 59 the conversion was raised to 64% for 70 days to determine the effect on the run. The conversion level was raised to 66% for 40 days, lowered for 5 days to 64%, and raised to 65% for 70 days with no significant influence on the flow coefficient. The conversion level was then lowered to 58% for 5 days and returned to 65% for 20 days (the remainder of the run). The run was terminated at 343 days due to tube metal temperatures (TMT). For example, if the TMT is greater than or equal to 1080° C. (1976° F.) as measured by an optical pyrometer, the furnace should enter the decoking process.

The Coil Flow Coefficient 604 showed a cresting point 608 (i.e., a change in curvature) at around day 10-11 of the experiment shown in FIG. 6, after which the Coefficient 604 essentially leveled off until the conversion level was increased. This result suggested that a hold time of about 10-11 days was all this furnace style required. On the next run, the furnace was started up at 58% conversion and held for 11 days. The conversion was then slowly raised to 65%. This furnace remained online for more than 350 days.

The experiment depicted in FIG. 6 showed the benefit of utilizing the coil flow coefficient to determine an appropriate predetermined interval for the first conversion level. The Darcy-Weisbach equation normalizes the difference in pressure as it compares to the flow, molecular weight and temperature, demonstrating that the heavy molecular weight materials were not accumulating on the tube walls as previously seen. The experiment indicated a large potential benefit in increased production days by reducing days the furnace is offline for decoking procedures. The experiment also showed an improved ethylene yield in the furnace effluent when measured over the entire run.

EOIs were also conducted on two furnace technologies, Foster Wheeler 11-pass coil design and Stone and Webster 4-pass coil design, across nine different furnaces, and it has been observed that these results are proportionately the same on a 4-pass as an 11-pass coil furnace.

On a Foster Wheeler 11-pass 460′ coil furnace, it took 17 days at 58% conversion for the furnace to arrive at the cresting point. The conversion was then raised over 10.5 days to 65%. The run length was between 450 days and 560 days.

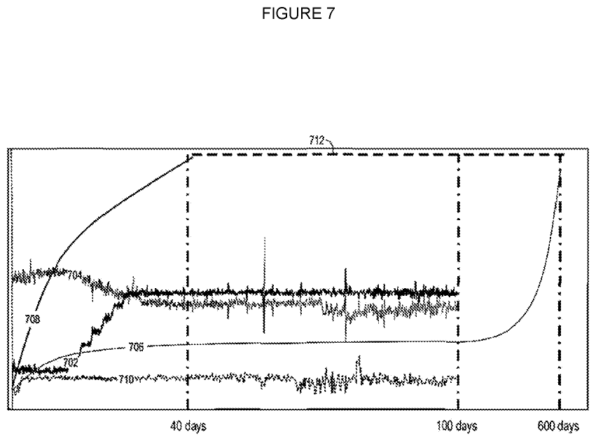

FIG. 7 shows a graph for an experiment performed on a furnace having four coils, with conversion 702, actual ethylene make (mol % kg/hr/coil) 704, Coil Flow Coefficient 706, Comparative Coil Flow Coefficient 708, Furnace Outlet Pressure 710, and Maximum Coefficient Limit 712. Maximum Coefficient Limit 712 refers to the maximum limit the Coil Flow Coefficient can reach before the furnace must be taken offline for decoking. Conversion 702 starts at a first conversion level 58%, which was maintained for 17 days, and was then gradually ramped to a second conversion level of 65%. The fouling rate remained relatively low throughout the run, as shown by Coil Flow Coefficient 706. Comparative Coil Flow Coefficient 708 refers to the fouling rate for a comparative method where the furnace is started up at an initial conversion level of 65%. As can be seen in FIG. 7, the Coil Flow Coefficient 708 of the comparative method is significantly higher, and reached the Maximum Coefficient Limit 712 after about 40 days, which required the furnace be taken offline for decoking. In contrast, the example method did not reach the Maximum Coefficient Limit 712 until about 600 days, allowing for an extended furnace run length.

Other implementations are also within the scope of the following claims.

1. A method comprising:
starting up a hydrocarbon cracking furnace at a first conversion level of a C₁-C₄ alkane, wherein the first conversion level is less than or equal to 60% conversion; and
maintaining the first conversion level for a predetermined interval,
wherein a run length of the furnace is at least 90 days.

2. The method of claim 1, wherein the predetermined interval is between about 1 day and about 75 days.

3. The method of claim 1, wherein the predetermined interval is at least 3 days.

4. The method of claim 1, wherein the first conversion level is in a range of about 50% to about 58%.

5. The method of claim 1, wherein the first conversion level is in a range of about 55% to about 58%.

6. The method of claim 1, wherein the C₁-C₄ alkane comprises ethane.

7. The method of claim 1, wherein the C₁-C₄ alkane is converted to ethylene.

8. The method of claim 1, further comprising increasing the first conversion level of the C₁-C₄ alkane to a second conversion level, wherein the second conversion level is greater than or equal to about 61%.

9. The method of claim 8, wherein increasing the first conversion level to the second conversion level comprises iterating between:
increasing the first conversion level by about 0.5 to about 2% over a period of time in a range of about 2 hours to about 24 hours; and
holding for a hold time in a range of about 12 hours and about 36 hours.

10. The method of claim 8, wherein the second conversion level is in a range of about 61% to about 75%.

11. The method of claim 8, wherein the second conversion level is about 65%.

12. (canceled)

13. The method of claim 8, wherein a run length of the furnace is at least 150 days.

14. The method of claim 8, wherein a run length of the furnace is at least 400 days.

15. The method of claim 8, wherein a run length of the furnace is extended by at least 25% as compared to a run length of the method performed without starting up at the first conversion level.

16. The method of claim 8, wherein a run length of the furnace is extended by at least 100% as compared to a run length of the method performed without starting up at the first conversion level.

17. The method of claim 8, wherein a run length of the furnace is extended by at least 200% as compared to a run length of the method performed without starting up at the first conversion level.
